(19) United States
(12) Patent Application Publication
(10) Pub. No.: US 2004/0163188 A1
(43) Pub. Date: Aug. 26, 2004

(54) Formulation and process for determining hair color

(76) Inventors: Rex Anthony Firkins, Coronado, CA (US); Steven John Gray, Coronado, CA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/368,507

(22) Filed: Feb. 18, 2003

(51) Int. Cl. A61K 7/13; A61K 7/06; G06F 19/00; G01N 33/48; G01N 33/50

(52) U.S. Cl. 8/406; 702/19

(57) ABSTRACT

A system and method for formulation of a personalized permanent hair color product. The system and method are used to produce on-site at the salon or home, a permanent, semi permanent and/or stained hair color custom mixed to according to features and individual parameters for each person, based upon an assessment test completed by the individual or a professional, based upon the person's “map” and characteristics. The person is then able to choose the quadrant of color (i.e. light blonde, blonde, red, brown, dark brown), and receive a unique color based upon his or her own “colormap”. Colormapping cannot only determine what colors work best with an individual's features, but also how best to structure it.

### BACKGROUND OF THE INVENTION

The present invention relates generally to personalized hair coloring compositions formulated based on a colormapping assessment test; and more particularly, to a method and system used to produce on-site, e.g., at a salon or home, a personalized permanent, semi permanent, or stained hair color in accordance with the colormap and the desire of the person for whom the hair color is produced. The key to choosing the correct mixture of color is based upon the colormap.

Inside the cortex of the hair fiber is the substance called melanin. Melanin gives the hair its natural color. It is the pigment of the hair. Two general classes of such pigments have been identified: eumelanins (characteristically brownish black) and pheomelanins (characteristically reddish orange). The concentration and combination of these two types of pigments are responsible for the natural color of hair. Dark hair has a higher concentration of the eumelanin, while red hair contains more pheomelanin. Light hair has reduced levels of both.

As hair ages, the pigments begin to break down under the natural influence of the environment, such as from exposure to sun or smoke and like pollutants. Also, in many individuals, the natural color of the hair may discolor with age or illness and develop an undesirable tone even without chemical processing or environmental exposure. As a result, hair may lose its natural brilliance over time. Additionally, in many people, hair follicles produce less and less pigment, as a person gets older. As a result, new growth of hair may be gray or white. The graying of hair is typically a gradual process, and not all hair may turn gray at once.

The ability to change the appearance of one's hair through chemical processes is important to both men and women. More specifically, it is important for both men and women to be able to chemically relax or to permanently wave the hair so that they may keep up with the rapidly changing hairstyle fashion in the hair industry. In some instances, such chemical processes, and alkali hair relaxers in particular, have the undesirable and very noticeable effect of discoloring of the natural underlying color of hair, particularly naturally blonde and gray hair.

For many people, the discoloration and graying of their hair is undesirable. As a result, many people seek to cover up or reduce the appearance of gray in their hair. These people will typically color their hair with a hair color formulation intended to deposit color on their hair. In many people, gray, or white un-pigmented hair is blended in with that person's naturally pigmented hair and it is impractical to deposit color only on the un-pigmented portions of their hair. As a result, these people require a hair color formulation to both cover up the un-pigmented hair and add color to their pigmented hair.

Traditional hair color systems and their method of use are based on levels (or depth) and tones. Typically, several stock formulations are provided that vary in the shade. A colorist in a salon, referring to memory or by comparing to a set of standard swatches, will determine the natural base level of the clients hair, determine the level they wish to achieve and determine the tone desired. The colorist will then chose the appropriate shade (or mix of shades) from the given stock of permanent color alternatives, or palette. The various shades in the stock palette of permanent hair colors are not formulated based on individual's colormap.

There exists a need, therefore, for a system and a method of preparing appropriate hair color formulations on-site tailored to the colormap of each customer's hair, which will produce a hair coloring formula precisely tailored to the customer's hair color, tone and proportion of gray, which eliminates the inventory and stock problems associated with the current practice, which will permit the colorist a high degree of creative control in addressing the gamut of hair tones and colorings which will be encountered in practice, and which will address the ad-hoc experimentation of the current practice to mix personalized hair color formulations from stock offerings.

There are known in the prior art various industry-available dye color mixing applications. These applications are typically directed to the formulation of the color dyes themselves at a plant or factory for their use in the actual salon hair coloring process. For instance, a number of industry applications are directed to allowing a colorist to cross-reference a client's hair color level against either a hair tone or a desired color level, so as to permit the colorist to select a single pre-mixed color dye from a large quantity of different pre-mixed dyes that are shipped by the manufacturer of the system for use with the system.

Typically, these dyes are then mixed with a so-called “generator” or “developer” to activate the color for the processing treatment. The colorist's choice of the single, appropriate pre-mixed dye product is sometimes governed by a chart which cross-references hair color level against hair tone or desired color level. The available pre-mixed dyes are listed at various locations on the chart, typically with a single, pre-mixed dye satisfying a small number of combinations, and often only one, of specific hair color level against desired hair color level or hair tone. Examples of such systems include the Logics International, Inc. “Color Facts” and “Attitudes” dye product-system; the Synaplex “Mid-Color” Ultra-Shine No-Lift Hair Color system; and the Wella “Color Charm” Liquid Cream Hair Color system.

Certain other color dye preparation systems provide the colorist with some degree of guidance to adapt certain particular pre-mixed dyes, or combinations thereof, so as to address a particular client's hair. These systems oftentimes extend to voluminous or otherwise cumbersome reference materials which must be utilized or cross-referenced in accordance with the base system. An example of such system is described in the Clairol Professional Encyclopedia, “Hair Color.”

For instance, the Clairol Encyclopedia refers to a so-called “Torrids” formulator chart cross-referencing a natural hair color against a desired hair color. The chart features a series of individual discrete boxes, each box representative of a single natural hair color level and a single desired hair color level, leading the colorist to select one or more hair dyes which can be used independently or mixed to some degree to meet that single natural hair color level and that single desired hair color level.

Separate from the chart, the Encyclopedia provides the colorist with certain textual recommendations for use with the Torrids system, in advising of a custom dye formulation table listing for the colorist certain customized formulas of two or more volumetric quantities of color dyes. However, these recommendations tend to be for specific coloring instances only and are not tied to any particular organized methodology to prepare color dye mixes for any type of possible hair coloring situation. Various aspects of color dye mixing is also referenced in the Schwarzkopf Color Technical Manual (albeit without the provision of a chart correlating hair colors and tones) and in the Framesi 2001 Hair Color Training Manual.

As will be appreciated, most of the existing color dye systems employ a large number of pre-mixed color dyes. Typically, it is the intent of these systems that the colorist select a single pre-mixed dye from a large number used in the particular coloring system. The intent of these color dye systems is to select a color dye which will change the color of a person's hair, cover gray hair, or the like. In effect, then, these systems endeavor to make a precise color selection for a very specific, narrow characteristic of a person's hair.

While certain of the dye systems include some background information as to combining more than one pre-mixed dye to accommodate certain enumerated particular hair coloring needs, the information is oftentimes not made integral with the bulk of the system and is provided on an ad hoc basis of specific examples and with no formal organization. In fact, the modification information is typically embodied in a textual form separate and apart from the depictions used in the system, and the information is oftentimes limited to a discrete number of particular coloring applications, without any particular methodology or organization to guide the colorist to modifications across a wide gamut of potential hair coloring situations. Ultimately, other than the several specific examples given, these systems give little guidance to the colorist in formulating a personalized hair color composition for an individual. Generally, then, owing to the existing concerns, these systems are not adapted to on-site preparation of a hair color product based on the colormap of the individual.

The prior art lacks a method and system that allows the hair colorist or individual to personalize the shade selection for a hair color formulation based on the client's own colormap, and desire. For the client, the resulting hair color should look more natural and for the colorist, the selection and the amount of mixing of the color for their client should be simplified and more precise. For the manufacturer, a limited number of shades can be organized into a system providing comprehensive coloring solutions covering the complete gamut of possible hair coloring requirements.

### SUMMARY OF THE INVENTION

A method for providing an individualized hair colorizing product for colorizing the hair of a selected individual, includes the steps providing a colormapping assessment test for assessing the color characteristics of a plurality of individuals and providing a corresponding plurality of sets of color parameters according to the assessing. First determining a colormapping relationship between: (i) each set of color parameters of the plurality of sets of color parameters, and (ii) a color mix of a plurality of color mixes is performed to provide a plurality of colormapping relationships and the plurality of colormapping relationships is stored at a first location. The colormapping assessment test is applied to the selected individual to provide a selected set of color parameters in accordance with the selected individual and first transmitting of the selected set of color parameters from a second location remote from the first location to the first location is performed. Second determining at the second location a color mix of the plurality of color mixes in accordance with the transmitted selected set of color parameters and the stored plurality of colormapping relationships is performed and second transmitting the selected color mix to a third location remote from the second location is performed to provide a transmitted color mix. Differing colorizing agents are mixed in accordance with the transmitted color mix to provide the individualized hair colorizing product and individualized hair colorizing product is applied to the hair of the selected individual.

The present invention thus includes a method of using a stock palette of hair color packets consisting of different tones and levels and intensifiers. In accordance with the invention, the palette of the stock permanent hair color formulas can be divided into at least two and preferably three different groups of tonal series. For example, Perfect Blondes, Perfect Reds, and Perfect Browns.

An advantage of the present invention allows the hair colorist to personalize the shade selection based on the client's colormap. For the client, the resulting hair color can look more natural and for the colorist, the selection and the amount of mixing of the color for their client is simplified.

Therefore, it is an object of the present invention to provide a system and method of formulating personalized hair-coloring compositions based on the colormap of a person.

It is another object of the present invention to provide a palette of stock hair color packets, to accomplish the individually custom mixed color It is another object of the present invention to provide a system and method for on-site formulation of hair color compositions for individual users to accommodate a wide variety of hair tones and colorings which can be easily modified by the colorist as desired, and which will yield a consistent product each time it is used that is appropriate for the individual's hair color and tone.

Still other objects and advantages of the invention will in part be obvious and will in part be apparent from the specification.

In accordance with the present invention, these and other objectives are achieved by a method and system of formulating personalized hair coloring compositions based on the colormap of the person; and more particularly, a method and system are used to produce on-site, e.g., at the salon or home, a personalized custom hair color in accordance with the original colormap of the person for whom the hair color is produced. In a preferred embodiment, the palette of stock hair color formulas is divided into five (5) different groups of tonal series.

The invention accordingly comprises the several steps and the relation of one or more of such steps with respect to each of the others, and the composition embodying the several elements and the combination of one or more of such elements, all as exemplified in the following detailed disclosure.

A system and method for formulation of a personalized permanent hair color product. The system and method are used to produce on-site at the salon or home, a permanent, semi permanent and/or stained hair color custom mixed to according to features and individual parameters for each person, based upon an assessment test completed by the individual or a professional, based upon the person's “map” and characteristics. The person is then able to choose the quadrant of color (i.e. light blonde, blonde, red, brown, dark brown), and receive a unique color based upon his or her own “colormap”. During the last few decades, more than at any other time in the world, as race and ethnic barriers have given way to openness, we have seen a world of color go from four individual seasons, to an explosion of variety. Colormapping cannot only determine what colors work best with an individual's features, but also how best to structure it. When used correctly, it can save both clients and stylists from the emotional and professional anguish that accompanies hair color, makeup, and clothing that is not right for that particular person. An individual can look completely natural and unique, in whichever color he chooses to go. If he wants blonde, there is a blonde just for him, that suits him perfectly, and that is unique to him. The same goes for any other color.

### DESCRIPTION OF THE DRAWING

The drawing is a block diagram representation of the color assessment process of the present invention.

### DETAILED DESCRIPTION OF THE PREFERRED EMBODIMENTS

The personalized permanent hair color in accordance with the invention is formulated from a hair color stock palette comprising at least 5 groups of tonal series which are selected and mixed in accordance with the colormap of the person for whom the formulation is made.

In a preferred embodiment, the hair color will be in the form of a creme gel having a viscosity that allows for brush/bowl or bottle application.

In a preferred embodiment, Color Swatch taps and labels specify the color tone for determining parts of the colormap. The same color tone tabs appear on the swatches in the explanatory materials and as labels on the packaging materials for easy identification.

As is well understood by those of ordinary skill in the art, hair color compositions work in combination with a developer. Preferably, the combination of hair color and developer will provide: superior conditioning benefits, a creamy spreadable consistency, a stay-put moist consistency (not too thin, not too thick) for client comfort, and an appropriate viscosity for optional, convertible bowl and brush or bottle application.

In a preferred embodiment, the developer also comprises several formulations to finely and precisely control the lifting and depositing action in the hair color formulations. Further, these several formulations should be completely intermixable, providing even greater flexibility.

For example, in a preferred embodiment, the developer is designated in accordance with the level of lift; for example, developer may be designated as 10, 20, 30, and 40 formulas, as described in Table I, below.

TABLE I

| | |
|---|---|
| 10 Volume Developer | Formulated to reduce the lightening |
| | action of the permanent hair color. 10 |
| | developer is used whenever minimized |
| | lifting action and increased color depth is |
| | desired. |
| 20 Volume Developer | Formulated as the standard developer |
| | for the permanent hair color. 20 |
| | developer is formulated to provide |
| | complete gray/white coverage and is |
| | used when standard lightening action |
| | and optimum color deposit and intensity |
| | are desired. |
| 30 Volume Developer | Formulated to increase the lightening |
| | action of permanent hair color. 30 |
| | developer is use when additional |
| | lightening action and optimum color |
| | deposit is desired. |
| 40 Volume Developer | Formulated for maximum lightening |
| | action of permanent hair color |

In a preferred example, the developers will vary in strength and concentration of hydrogen peroxide. Thus, 10 developer is 3% (by vol.) hydrogen peroxide, 20 developer is 6% (by vol.) hydrogen peroxide, 30 developer is 9% (by vol.) hydrogen peroxide, and 40 developer is 12% (by vol.) hydrogen peroxide.

The system and method of the present invention allows a colorist to formulate a personalized hair color based on the colormap for the person for whom the formulation is prepared. In use, the colorist first completes the Colormap for the person whose hair they are coloring.

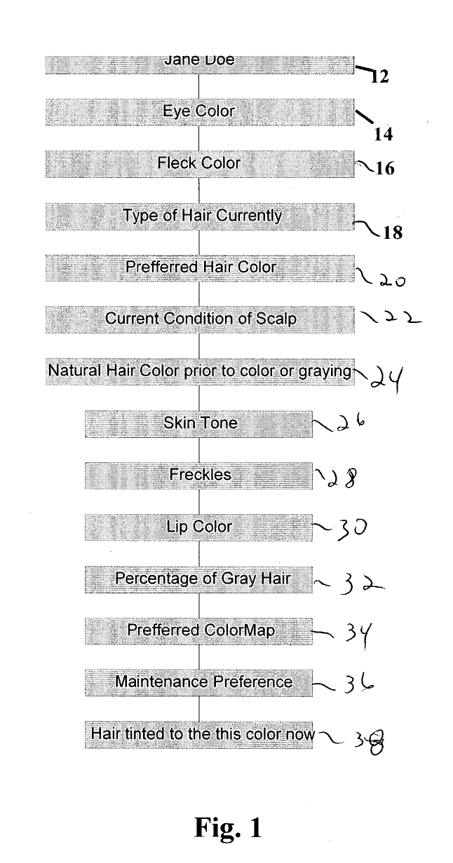

In order to do this the colorist administers the colormapping assessment test, an example of which is attached as Appendix A. The color assessment test can follow the steps of color-assessment process 10 in FIG. 1. The process proceeds from determining eye color in step 12 to determining flesh color, type of hair currently and preferred hair color in steps 14-20.

The current condition of the scalp is determined in step 20, and the natural hair color prior to color or graying, the skin tone, freckles and lip color are determined in steps 24-30 respectively. The percentage of gray hair is determined in step 32. The preferred embodiment and the maintenance preference are determined in steps 34, 36, respectively. In step 38 a determination is made how the hair is currently tinted.

An example of applying color assessment process 10 to a selected individual is shown in Appendix B. The results of the color assessment process 10 in Appendix B are shown as color assessment vector 50, shown in Table II.

TABLE II

Green
Blue
Virgin
Blond (light)
Normal
Medium Brown
Light
None
Peach
Slightly Gray 25%
Natural
High
Blond

The color assessment vector 50 is then used to access an individual mix which is ideal for the person for whom the color assessment vector 50 pertains as determined by the color assessment test of Appendix A. In order to do this the color assessment vector is applied to the color mapping matrix of Appendix C. The individual would then contact Colormapping Customer services, or whoever was in possession of these results, and receive the hair color formula, based on packet numbers. For instance, they could be given the following directions.

Mix together the following packets, which can typically be divided into 25 ml packets or 60 ml tubes, for your colormapping custom hair color: 1, 2, 3, 5, 7, 18, 24, 26, 27, 43, 46 and 50. The colormap also considers the effects of porosity and texture of the person's hair. The person would then be directed to follow the directions inside their kit for application.

Generally, an application time of thirty (30) to forty-five (45) minutes is appropriate, however, for super light blonde, a time of forty-five (45)-fifty (50) minutes is appropriate.

The process of applying the hair color/developer formulation amount is as follows:

(1) apply formula to the first one-half (½) inch section from the scalp;

(2) take one-quarter (¼) inch partings;

(3) mix formula for mid-shaft and apply;

(4) process thirty (30) to forty-five (45) minutes as appropriate;

(5) on overly porous ends, do not immediately work through ends;

(6) wait until hair is almost completely processed to avoid ends taking on too much tone;

(7) in applying retouch application with hair color: apply to new growth only;

(8) if necessary, when the processing time is almost through, replenish the previously colored hair.

In accordance with the invention disclosed are novel personalized hair coloring compositions and methods of formulating that use a stock palette of permanent, semi permanent, and or staining hair color formulas consisting of different tones and levels with intensifiers. The shades in the palette are formulated to create mixtures of stock based on the individuals “colormap”.

The palette of the stock permanent hair color formulas is divided into five (5) different groups of tonal series; Group 1 or “perfect blondes,” Group 2 or “perfect reds,” and Group 3 or “perfect browns”, Group 4, or “perfect highlights” Group 5, or “Colorstaining for Men”.

An advantage of the system of the invention is that the hair colorist personalizes the shade selection based on the client's own “colormap”. For the client, the resulting hair color will look more natural and for the colorist, the selection and the amount of mixing of the color for their client is simplified and more precise. The advantage of our method and system is that simplifies the selection of the proper shade for an individual by the hair colorist. A Table of Historic Features is included as Appendix D.

While the invention has been described in detail and with reference to specific examples thereof, it will be apparent to one skilled in the art that various changes and modifications can be made therein without departing from the spirit and scope thereof. It will thus be seen that the objects set forth above, among those made apparent from the preceding description, are efficiently attained and, since certain changes may be made in carrying out the above methods and in the devices as set forth without departing from the spirit and scope of the invention, it is intended that all matter contained in the above description shall be interpreted as illustrative and not in a limiting sense.

### Appendix A

### Colormapping Assessment Test

1. My natural eye color is

Blue

Green

Brown

2. The color of the flecks in my eye is

Blue

Green

Gold

Brown

Amber

3. What type of hair do you have currently?

Virgin

Natural Sun Bleached

Chemically Bleached

Permed

Straightened

Chemically Colored

4. My preferred hair color is the following

Blonde (light)

Warm (Amber, Copper and Red)

Medium Brown

Dark Brown

5. The current condition of my scalp is

Dry

Oily

Normal

Flaky

6. My natural hair color is

Black (very dark brown)

Dark Brown

Medium Brown

Red

Ash Blonde

Light Blonde

7. My skin tone is

Light

Fair

Medium

Dark

8. My freckles are best described as

None

Slightly

Moderate

Heavy

9. My natural lip color is

Peach

Pink

Plum

10. I have approximately this much gray hair:

None

Slightly Gray 25%

Moderately Gray 50%

Mostly Gray 75%

Totally Gray 100%

11. Take me to my colormap that is most

Natural

Dramatic (intensified warmth)

Glamorous (intensified browns)

12. I am comfortable maintaining as follows:

Minimum (every 8 weeks approximately)

Average (every 6 weeks approximately)

High (every 4 weeks approximately)

13. My hair is tinted to the following now:

Not tinted

Blonde

Light Brown

Warm

Dark Brown

Highlighted

### Appendix B

1. My natural eye color is

Green

2. The color of the flecks in my eye is

Blue

3. What type of hair do you have currently?

Virgin

4. My preferred hair color is the following

Blonde (light)

5. The current condition of my scalp is

Normal

6. My natural hair color is

Medium Brown

7. My skin tone is

Light

8. My freckles are best described as

None

9. My natural lip color is

Peach

10. have approximately this much gray hair:

Slightly Gray 25%

11. Take me to my colormap that is most

Natural

12. I am comfortable maintaining as follows:

High (every 4 weeks approximately)

13. My hair is tinted to the following now:

Blonde

[image]

[image]

[image]

[image]

[image]

[image]

[image]

[image]

[image]

[image]

[image]

[image]

[image]

[image]

[image]

APPENDIX D

| | SKIN | EYES | LIPS | HAIR |
|---|---|---|---|---|
| NORDIC | PINK, VIOLET | GRAY, VIOLET, BLUE | PINK, BLUE, | 6-9 LEVELS, BLONDE, ASH, GREEN, YELLOW |
| | | | PASTEL, NON- | |
| | | | EXISTENT | |
| IRISH | PALE WITH WARM | GREEN, SMOKY BLUE, GREEN | PURPLE, | |
| | FRECKLES | BROWN | ORANGE MELON, | |
| | | | BRIGHT PINK | |
| | | | SALMON | |
| BLACK IRISH | WHITE | ICE BLUE | STRAIGHT | FULL BODY, RE-DARK BROWN, DARK AUBURN, AND |
| | | | SHAPE, PINK | BLACK |
| EASTERN EUROPE- | FAIR, GOLDEN WITH | BROWN, BLACK, DARK BLUE, | MATTE | STRAIGHT, DARK BROWN, NOT BLACK |
| LEBANESE, RUSSIAN, | GREEN UNDERBASE | ALMOND SHAPE | | |
| SIBERIAN, MONGOLIAN, | | | | |
| IRANIAN | | | | |
| LATIN-HISPANIC | LIGHT CAFE, LIGHT AND | GREEN, HAZEL, BROWN RED, | PURPLE BROWN, | BROWN |
| | GOLDEN UNDERBASE, | NEVER BLUE | PINK BROWN | |
| | BRONZED, TROPICAL | | | |
| | OLIVE | | | |
| MEDITERRANEAN GREEK, | DARK OLIVE | | | |
| ITALIAN | | | | |
| GERMAN | PINK, FAIR, SALMON, | HAZEL, BLUE, GRAY, BROWN- | BRIGHT PINK, | LEVEL 3-6, BROWN, WARMER, PINCH OF BLACK |
| | YELLOW UNDERTONES | RED | PEACH | |
| FRENCH | DARK, MORE OLIVE PIGMENT | DARK RICH, GREEN | FULL OF COLOR | 2-4 LEVELS |
| | | INTENSITY | | |
| ENGLISH-WELCH | ASH UNDERTONES, PINK, LIGHT, | LIGHT GRAY, AQUA | | 7 LEVELS, LESS PIGMENT, LIMP, |
| | FAIR WITH VEINS | BLUE, TEAL | | TELLOW, GREEN, ASHY BROWN |
| AFRICAN | PURPLE, GREEN UNDERTONES | DARK RED, BLACK, | | CURLY |
| | | BROWN | | |
| EAST INDIA-PAKISTAN | GREEN UNDERTONES, DARK, FAIR, | RED, DARK BROWN | SMOOTH, SMOKY | FULL BODY, BLACK |
| INDIA | MIDTONE FRECKLES | | | |
| WEST EUROPEAN | PINKY, SLIGHT ASH, SALLOW WITH | GREEN | 4-8 LEVELS, YELLOW, | |
| | BLUE UNDERTONES | | NEUTRAL, 6 LEVEL-AUBURN, | |
| | | | BROWN, GREEN | |
| NORTH AMERICAN INDIAN | BROWN, RED | DARK RED, BROWN, | ORANGE, TERRACOTTA | STRAIGH, BLACK |
| | | BLACK | | |
| ASIAN | YELLOW, GREEN UNDERTONE, | BLACK, BROWN | THIN AND NEUTRAL IN COLOR | −4 LEVELS, COARSE, STRAIGHT, |
| | FAIRISH | | | BLACK, BROWN |

What is claimed is:

1. A method for providing an individualized hair colorizing product for colorizing the hair of a selected individual, comprising the steps of:
(a) providing a colormapping assessment test for assessing the color characteristics of a plurality of individuals and providing a corresponding plurality of sets of color parameters according to said assessing;
(b) first determining a colormapping relationship between: (i) each set of color parameters of said plurality of sets of color parameters, and (ii) a color mix of a plurality of color mixes to provide a plurality of colormapping relationships;
(c) storing said plurality of colormapping relationships at a first location;
(d) applying said colormapping assessment test to said selected individual to provide a selected set of color parameters in accordance with said selected individual;
(e) first transmitting said selected set of color parameters from a second location remote from said first location to said first location;
(f) second determining at said second location a color mix of said plurality of color mixes in accordance with said transmitted selected set of color parameters and said stored plurality of colormapping relationships;
(g) second transmitting said selected color mix to a third location remote from said second location to provide a transmitted color mix;
(h) mixing differing colorizing agents in accordance with said transmitted color mix to provide said individualized hair colorizing products; and
(i) applying said individualized hair colorizing product to the hair of said selected individual.

2. The method for providing an individualized hair colorizing product of claim 1, wherein said colormapping assessment test comprises a determination of a natural hair color.

3. The method for providing an individual hair colorizing product of claim 2, wherein said colormapping assessment test comprises a determination of a preferred hair color.

4. The method for providing an individualized hair colorizing product of claim 3, wherein the step of applying said colormapping assessment test further comprises the step of determining a natural eye color.

5. The method for providing an individualized hair colorizing product of claim 4, wherein the step of applying said colormapping assessment test further comprises the step of determining skin and scalp information.

6. The method for providing an individualized hair colorizing product of claim 1, wherein said first determining said colormapping relationships comprises the further steps of: (paragraph to be defined later).

7. The method for providing an individualized hair colorizing product of claim 1, wherein the step of first transmitting said selected set of color parameters further comprises the further step of transmitting by way of the internet.

8. The method for providing an individualized hair colorizing product of claim 1, wherein said first location is the same as said third location.

9. The method for providing an individualized hair colorizing product of claim 8, wherein said first location comprises a hair salon.

10. The method for providing an individualized hair colorizing product of claim 8, wherein said first location comprises a home of said selected individual.

11. The method for providing an individualized hair colorizing product of claim 1, wherein said colorizing agents comprise differing color tones, levels and intensities.

12. The method for providing an individualized hair colorizing product of claim 11, wherein said color tones comprise a plurality of groups of total series.

13. The method for providing an individualized hair colorizing product of claim 1, comprising the further steps of:
(j) applying a colormapping assessment test to said selected individual to provide a further selected set of color parameters; and
(k) repeating steps (e)-(i) in accordance with said further selected set of color parameters.

14. The method for providing an individualized hair colorizing product of claim 1, wherein step (d) comprises the further step of applying said colormapping assessment test to said selected individual by way of a telephone line.

15. The method for providing an individualized hair colorizing product of claim 1, wherein step (d) comprises the further step of applying said hair colormapping assessment test to said selected individual by way of the internet.
